(19) United States
(12) Patent Application Publication
(10) Pub. No.: US 2006/0107949 A1
(43) Pub. Date: May 25, 2006

(54) Holder for a dispensing container system

(76) Inventors: Michael Birsha Davies, Ware (GB); James William Godfrey, Ware (GB); Pal Kenneth Rand, Ware (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/533,827

(22) PCT Filed: Nov. 5, 2003
(86) PCT No.: PCT/EP03/12508

§ 371 Date: May 3, 2005

(51) Int. Cl. A61M 11/00; A61M 15/00

(52) U.S. Cl. 128/200.23; 128/203.15

(57) ABSTRACT

A holder for holding a dispensing container system which is adapted to dispense a quantity of a fluid contained therein on movement thereof relative to the holder and further includes a dispensing counter means for counting the number of quantities of the fluid dispensed is disclosed. The holder has a moulded plastic body with inner and outer surfaces. The inner surface bounds a cavity adapted to receive the dispensing container system in movable relation thereto. The cavity has a moulded counter advance means which is adapted in use to engage the dispensing counter means on relative movement between the dispensing container system and the body to advance the dispensing counter means to indicate the dispensing of a quantity of the fluid.

### RELATED APPLICATIONS

This International patent application claims priority from UK patent application No. 0226020.6 dated 7 Nov. 2002 and is related to the Applicant's concurrently filed International patent applications which respectively claim priority from UK patent application No. 0226021.4 dated 7 Nov. 2002 (Applicant's Ref: P33143) and UK patent application No. 0226022.2 dated 7 Nov. 2002 (Applicant's Ref: P33144). The entire content of each of these applications is hereby incorporated herein by reference.

### FIELD OF THE INVENTION

The present invention relates to a holder for holding a dispensing container system which is adapted to dispense a quantity of a fluid contained therein on movement thereof relative to the holder, and is particularly, but not exclusively, concerned with a holder for holding an aerosol canister.

### BACKGROUND OF THE INVENTION

A holder for an aerosol canister which acts as an actuator for the canister on relative movement therebetween is known in the field of inhalation devices, for instance the nasal inhaler marketed under the trade name Beconase® (GlaxoSmithKline) in which the aerosol canister contains a drug (beclomethasone dipropionate) formulated in a pressurised fluid propellant.

In the Beconase® device the aerosol canister has a canister body holding the drug formulation which is capped with a valve assembly comprising a valve stem through which the drug formulation is selectively discharged from the canister body. The valve stem is biased to an extended (closed) position relative to the canister body by a spring or the like in the valve assembly. In the extended position the valve stem places the drug formulation in fluid communication with a metering chamber in the valve assembly. This is the metering state and the metering chamber is sealed from the external environment in this state. The valve stem is movable against the biasing force to a retracted (open) position relative to the canister body. In the retracted position the valve stem places the metering chamber in fluid communication with the external environment whereby the metered quantity of the drug formulation in the metering chamber is exhausted from the canister for administration to the patient. This is the discharge state and in this state the metering chamber is sealed from the interior of the canister body.

The holder has a hollow tube-like container part with a cavity which receives the canister therein. As known in the art, a base of the cavity presents a hollow stem block to receive the valve stem in stationary relation thereto. An outlet nozzle sized to be received in a patient's nostril extends outwardly from the outer surface of the container part. The stem block has an orifice which is oriented towards the nozzle.

In use, the patient depresses the canister into the cavity of the container part thereby resulting in the canister body moving relative to the valve stem to bring the valve stem to its retracted position. This causes a metered amount of the drug formulation to be discharged from the valve stem and directed out of the nozzle by the stem block to be delivered to the patient's nostril.

In addition to the container part, the holder has a closure part which is hinged to the mouth area of the cavity of the container part so as to be hingeable from a closed position, in which the closure part cooperates with the container part to enclose the canister and the nozzle, to an open position in which the closure and container parts are arranged “back-to-back” to expose the canister and nozzle. In the closed position the holder acts as a protective casing for the inhalation device, while in the open position the patient can use the holder as an applicator for gripping the inhalation device when administering the drug.

A drawback of such an inhalation device is that there is no dose counter to count the number of doses of the drug delivered to the patient so that the number of doses left is known.

It would be useful to provide a holder of the type defined which is adapted to advance a dose counter on relative movement of the dispensing container system thereto.

### SUMMARY OF THE INVENTION

According to the present invention there is provided a holder for holding a dispensing container system which is adapted to dispense a quantity of a fluid contained therein on movement thereof relative to the holder and further includes a dispensing counter means for counting the number of quantities of the fluid dispensed, the holder having a moulded plastics body with inner and outer surfaces, the inner surface bounding a cavity adapted to receive the dispensing container system in movable relation thereto, the cavity having a moulded counter advance means which is adapted in use to engage the dispensing counter means on relative movement between the dispensing container system and the body to advance the dispensing counter means to indicate the dispensing of a quantity of the fluid, the body being formed with an outlet port in communication with the cavity such that the fluid dispensed from the dispensing container system is dischargeable therethrough, and an aperture extends through the body from the outer surface to the inner surface in alignment with the counter advance means.

The aperture is created in the moulding process by that part of the mould which forms the counter advance means. The aperture provides the means by which the mould part is extracted from the body after it has been moulded. This is particularly advantageous where the counter advance means is unable to be formed by a mould part extending through the outlet port, for example due to the size of the outlet port as in the case where the outlet port is in the form of a nozzle sized to be received in a nostril of a nose of a human patient, and/or where the counter advance means is non-aligned with the outlet port.

The counter advance means may be a rack-like means, for instance a rack-like member or post.

In an embodiment of the invention, such as hereinafter described, the cavity extends along an axis of the holder and the dispensing container system is axially, slidably movable in the cavity to dispense therefrom. The body may be adapted to receive the dispensing container system in the cavity only when it is arranged in a predetermined orientation about an axis thereof. The axis of the dispensing container system preferably extends between a trailing end and a leading end of the dispensing container system. The counter means may be located at the leading end and the body adapted to prevent rotation of the counter means in the cavity from the predetermined orientation.

Preferably, the body is provided with an alignment feature for aligning the system in the predetermined orientation. As an example, the alignment feature may be a slot in the body along which the system is slidable, as in the embodiment hereinafter described. Moreover, the slot may be adapted to engage the counter means to prevent its rotation in the cavity.

In an embodiment of the invention, such as hereinafter described, the counter means has a display part for displaying indicia to indicate the count and the body has a window for registering with the display part. The window may form part of the alignment feature, e.g. the slot at least in part extends from the outer surface to the inner surface. Conveniently, it is the display part which is slidable in the slot and/or which the slot engages for anti-rotation of the counter means.

The holder may have a hollow stand structure with a passageway in fluid communication with the outlet port, an outlet member of the container system through which fluid is dispensed being receivable in the passageway so that the passageway is able to channel fluid dispensed from the outlet member to the outlet port. The passageway may have an entrance opening into which the outlet member is insertable, and an exit opening aligned with the outlet port. The exit opening may be spaced from the outlet-port by a void area, as in the embodiment hereinafter described.

In an embodiment of the invention, such as hereinafter described, the counter advance means and/or stand structure is located on a base surface of the cavity. The counter advance means may be positioned to a side of the stand structure.

Preferably, the body is formed by injection moulding.

According to the invention there is further provided the holder of the invention and the dispensing container system or the counter means thereof. The system may comprise a container and the counter means. The container and counter means may be secured, fixedly or releasably, to form a unitary structure, or independent parts. In the latter case, the counter means may be mounted in the cavity, e.g. permanently.

The container may have an outlet member and a container member which contains the fluid and is movable relative to the outlet member to dispense the fluid from the outlet member. The container system may dispense when the container member is moved relative to the outlet member and the stand structure may be adapted to hold the outlet member stationary relative to the body so that the container member is movable in the body relative to the outlet member.

Ideally, the outlet member is at the leading end of the container system.

As in the embodiment hereinafter described, the container may be an aerosol container, in which case the outlet member may form a part of a valve mechanism secured to the container member which is selectively openable by movement of the container member in the body relative to the outlet member.

Preferably, the container system has a metering mechanism for dispensing metered doses of the fluid and the dispensing counter means is adapted to count the number of doses dispensed. To this end, the valve mechanism may be a metering valve.

The fluid in the dispensing container system may be a drug or pharmaceutical composition, e.g. a suspension or solution of a drug in a liquified gas propellant, such as HFA 134a or HFA 227. The drug may be for the treatment or prophylaxis of a respiratory disease or disorder, for instance rhinitis.

The present invention-further provides a moulding process for the holder of the invention which has the steps of providing a mould assembly for moulding the holder which includes a mould part adapted in use to form the counter advance means and the aperture in the moulded holder, moulding the holder in the mould assembly and disassembling the mould assembly from the holder with the mould part being extracted from the holder through the aperture.

A non-limiting exemplary embodiment of the invention will now be described with reference to the accompanying FIGURES of drawings.

### BRIEF DESCRIPTION OF THE DRAWINGS OF THE EXEMPLARY EMBODIMENTS

FIG. 1 is a schematic view of a patient using an intranasal device having an inner actuating part in accordance with the invention.

FIG. 2 is a side view of the intranasal device showing an outer casing part thereof comprising cover and container members and a hinge therebetween, the outer casing part in a closed state to protect inner parts of the device.

FIG. 3 is a side view of the intranasal device with the outer casing part hinged to an open state to allow access to the inner parts.

FIG. 4A is a side view of the intranasal device in its open state with the cover member pivoted behind the container member

FIG. 4B corresponds to FIG. 4A, but with the container and cover members nested.

FIG. 5 is a rear view of the device with the outer casing part in its closed state.

FIG. 6 is an exploded perspective view of the intranasal device with the outer casing part in its nested state.

FIG. 7 is a schematic, partial sectional side view of the inner actuating part of the intranasal device mounted in the outer casing part.

FIG. 8 is a enlarged fragmentary view of an alternative hinge construction for the outer casing part.

FIG. 9 is a cross-sectional side view of the alternative hinge construction along line IX-IX in FIG. 8.

FIG. 10A is a perspective view of a canister unit of the intranasal device comprising a canister and a dose counter head mounted on the canister.

FIG. 10B is a plan view of the dose counter head.

FIG. 10C is a side view of the dose counter head.

FIG. 10D is a rear view of the dose counter head.

FIG. 11 is schematic side view, partly in section, of the inner actuating part of the intranasal device.

FIG. 12 is an opposite side view of the inner actuating part with the canister unit shown mounted therein in ghost.

FIG. 13 is a plan view of the inner actuating part.

FIG. 14 is a rear view of the inner actuating part with the canister unit mounted therein.

FIG. 15A is a schematic side view, partly in cross section, of the canister unit mounted in the inner actuating part in an inoperative position.

FIG. 15B corresponds to FIG. 15A, but with the canister unit in an operative position relative to the inner actuating part.

### DETAILED DESCRIPTION OF THE EXEMPLARY EMBODIMENT

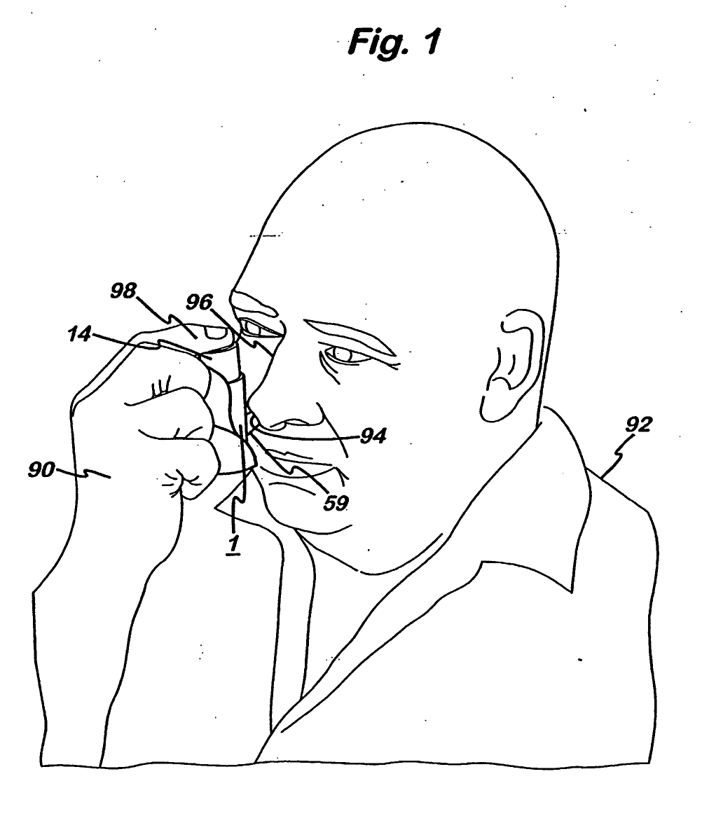

In the FIGURES of drawings there is shown a drug delivery device 1 in accordance with the present invention, the device 1 in this particular non-limiting embodiment being an intranasal drug delivery device, as shown graphically in FIG. 1.

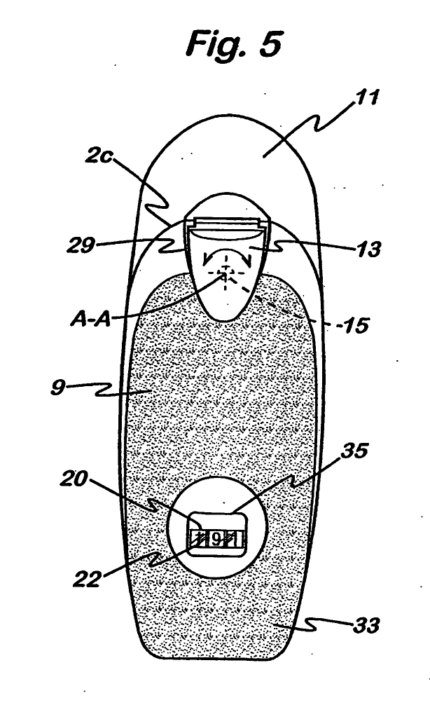

As best shown in FIG. 6, the intranasal drug delivery device 1 comprises as component parts the following:—
an outer casing part 3;
a one-piece inner actuating part 5 which is releasably securable in the outer casing part 3; and
a canister unit 7 releasably securable in the inner actuating part 5.
   Outer Casing Part

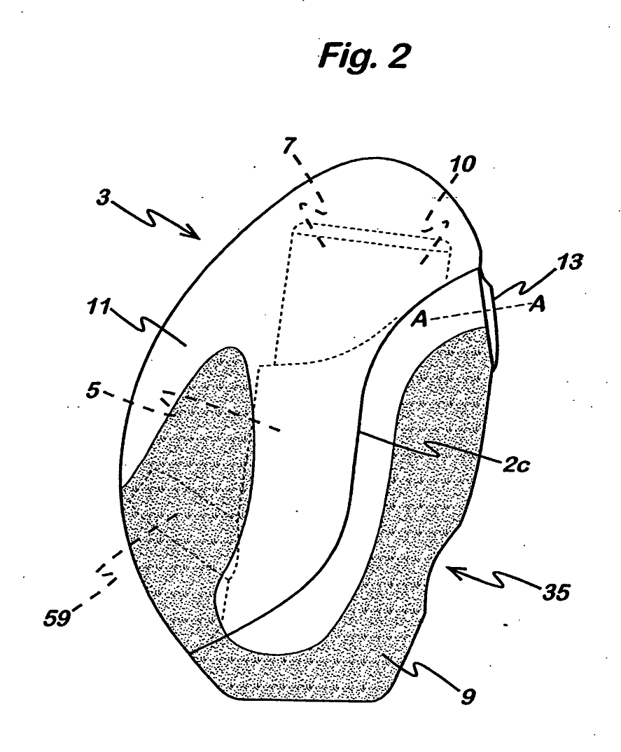

Referring to FIGS. 2 to 7, the outer casing part 3 of the intranasal device 1 is formed from two shell-like members, namely a container member 9 and a cover member 11, which are connected to one another through a hinge 13. The container and cover members 9, 11 each respectively present a cavity 17a; 17b having a mouth bound by a lip surface 2a; 2b (FIG. 6).

Both the container member 9 and the cover member 11 are formed from a plastics material by injection moulding, although other types of moulding processes can, of course, be used. The container and cover members 9, 11 are preferably both formed from polypropylene with the container member 11 being opaque, but with the cover member 11 being transparent or semi-transparent. Other plastics material combinations are possible, although it is preferable for the cover member 11 to be transparent/semi-transparent for reasons which will become apparent hereinafter.

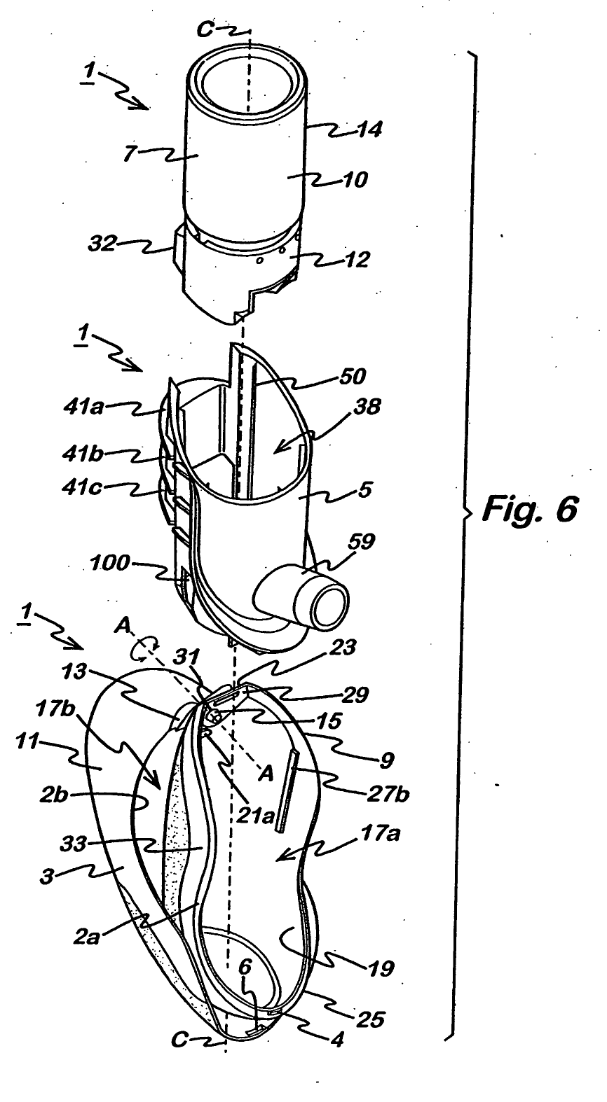

As shown in FIG. 7, for example, the cover member 11 is integrally formed with the hinge 13, colloquially known as a “living hinge”, and the hinge 13 includes a knob 15.

Turning to FIGS. 6 and 7, on an inner surface 19 of the cavity 17a of the shell-like container member 9 there is formed a pair of protrusions or ribs 21a, 21b to co-operate with complementary surfaces of the inner actuating part 5 to form a snap-fit connection between the outer casing part 3 and the inner actuating part 5, as will also be discussed in more detail hereinafter. A first one of the ribs 21a is disposed towards an upper end 23 of the container member 9, whereas the other rib 21b is disposed towards a lower end 25 of the container member 9.

Also formed on the inner surface 19 of the cavity 17a of the container member 9 are longitudinally extending ribs 27a, 27b. The longitudinal ribs 27a, 27b are disposed on opposing sides of the container member 9 and act as anti-rotational retainers on the inner actuating part 5 in the cavity 17a.

At the upper end 23 of the container member 9 there is formed a bevel 29 through which an aperture 31 extends from an outer surface 33 of the container member 9 to the inner surface 19 of the cavity 17a. From FIG. 7 it will be seen that the aperture 31 is adapted to receive the knob 15 on the hinge 13. Although the diameter of the knob 15 is greater than the diameter of the aperture 31, the knob 15 is able to be pushed through the aperture 31 for capture therein due to the container and cover members 9, 11 being sufficiently resiliently deformable due to their shell-like nature and the materials used. Moreover, the knob 15 is able to be withdrawn from the aperture 31 upon application of a sufficient pulling force thereto for separation of the container and cover members 9, 11.

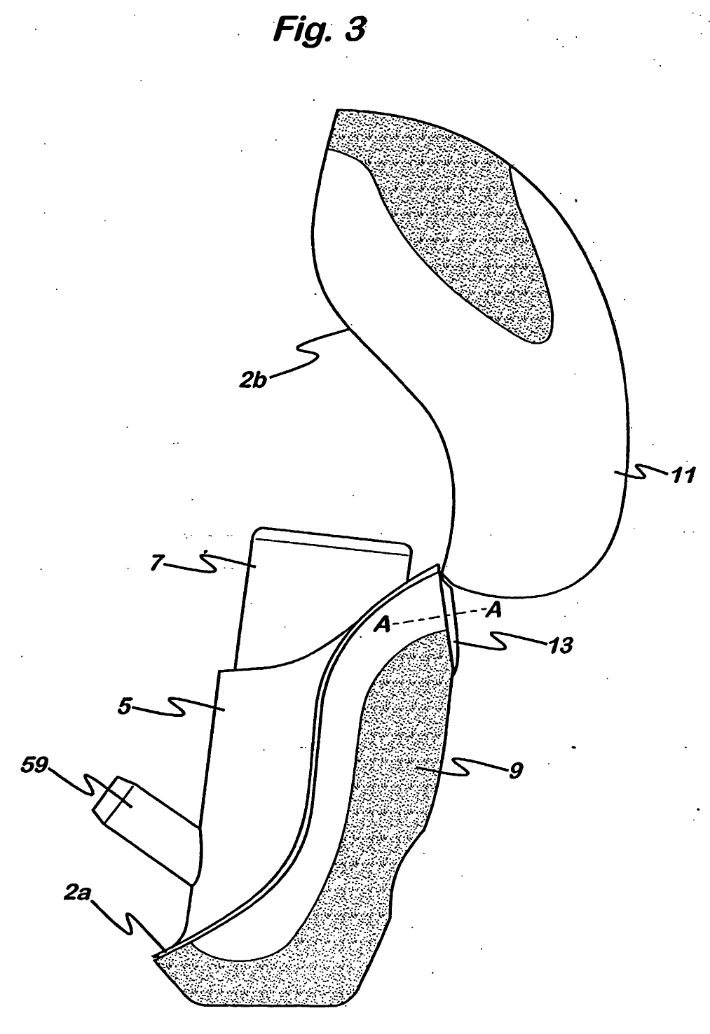

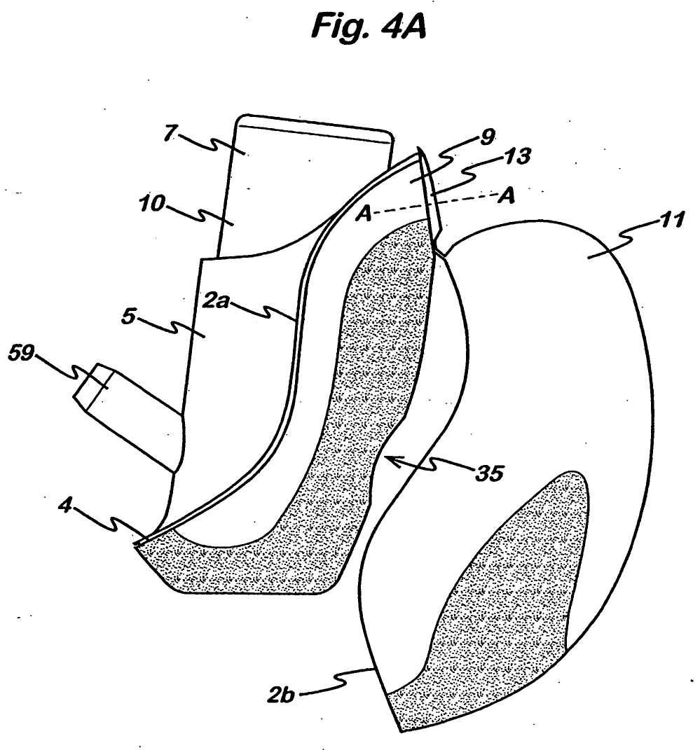

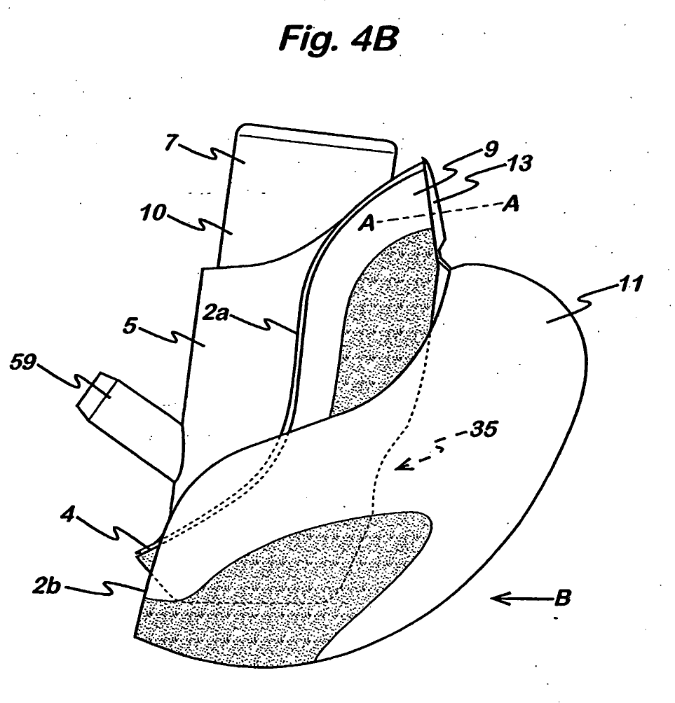

As shown in FIGS. 2 to 5, the mounting of the knob 15 of the hinge 13 in the aperture 31 of the container member 9 enables two degrees of movement of the cover member 11 on the container member 9 as follows:—
(1) Hinging of the cover member 11 from a closed position shown in FIGS. 2 and 5, in which the respective lip surfaces 2a, 2b of the cavities 17a, 17b of the container and cover members 9, 11 abut one another at an interface 2c to form an enclosed internal space 8 (see FIG. 7), to an open position shown in FIG. 3, in which the cavity 17a of the container member 9 is accessible. As will be understood from FIGS. 6 and 7, the lip surfaces 2a, 2b are each provided with detent elements 4, 6 which engage with one another in the closed position to provide a releasable snap-fit fastening of the container and cover members 9, 11 in the closed position.
(2) Rotary or pivotable movement of the cover member 11 about the aperture 31 (pivot axis A-A) from a first angular position corresponding to the open position shown in FIG. 3 to a second angular position shown in FIG. 4A in which the cover member 11 is located behind the container member 9. The cover member 11 is not able to be pivoted about the pivot axis A-A from its closed position due to the interengagement of the lip surfaces 2a, 2b.

As shown in FIG. 4B, when the cover member 11 is in the second angular position it is able to nest with the container member 9 by forward movement in the direction of arrow B. In other words, the concave cavity 17b of the shell-like cover member 11 is able to slidingly receive the convex rear surface of the container member 9. In the nesting position, an interference fit is formed between the container and cover members 9, 11 to releasable fasten them in the nesting position.

When the container and cover members 9, 11 adopt the nesting configuration, an ergonomic unit is formed which is able to be easily, and comfortably, held by a hand 90 of a patient 92, as shown in FIG. 1. In this mode, the outer casing part 3 is able to act as a holder or applicator of the drug delivery device 1.

As shown in FIG. 5, for example, the outer surface 33 at the rear side of the container member 9 is provided with a window 35. As will be understood from FIGS. 4A and 4B, the window 35 is covered by the cover member 11 when in its second angular position. If the cover member 11 is transparent or semi-transparent, the window 35 is then visible through the cover member 11 when in its second angular position.

If need be, an indexing or detent mechanism could be provided for indexing the cover member 11 in one or more predetermined angular positions about the pivot axis A-A, for instance the first and/or second angular positions and/or angular positions therebetween. The user would then have a tactile feedback indicating that the cover member 11 is in the correct angular position, e.g. for nesting with the container member 9. One way of achieving the indexing mechanism would be a male-and-female arrangement in which male (or female) surface features are formed in the outer surface 33 of the container member 9 at the required angular dispositions about the pivot axis A-A and one or more complementary female (or male) surface features are correspondingly arranged oh the inner surface of the hinge 13 about the knob 15. When the male feature is located in the, or one of the, female features (or vice-versa), the cover member 11 is indexed in a predetermined angular position. To move the cover member 11 to a new angular position, rotation of the cover member 11 about the pivot axis A-A causes disconnection of the first indexing connection until the male and female features reengage at a new angular position of the cover member 11

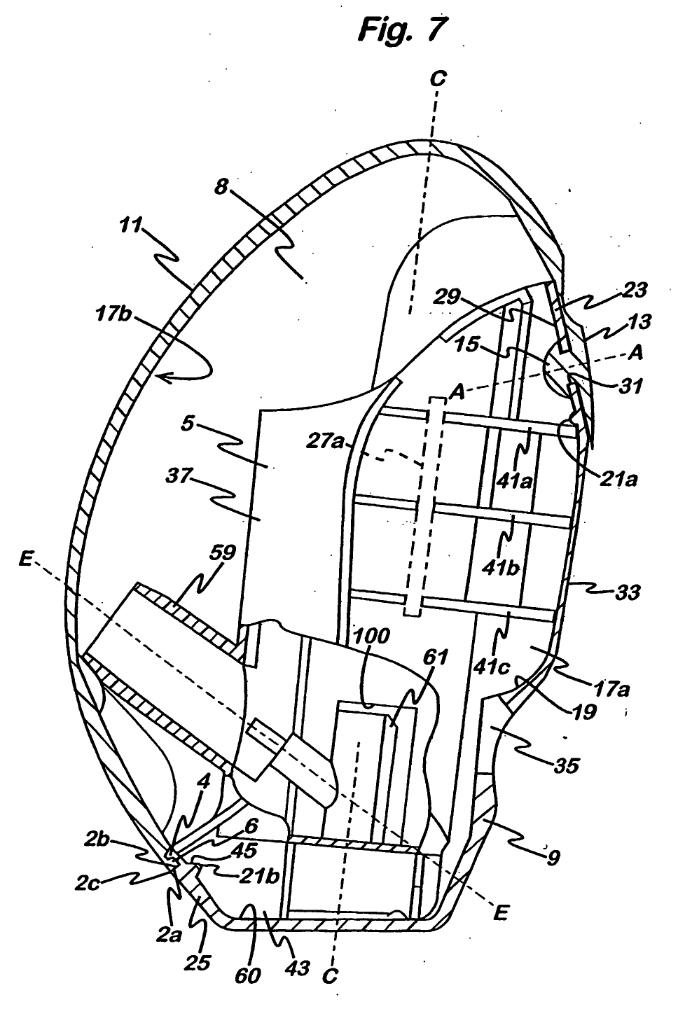

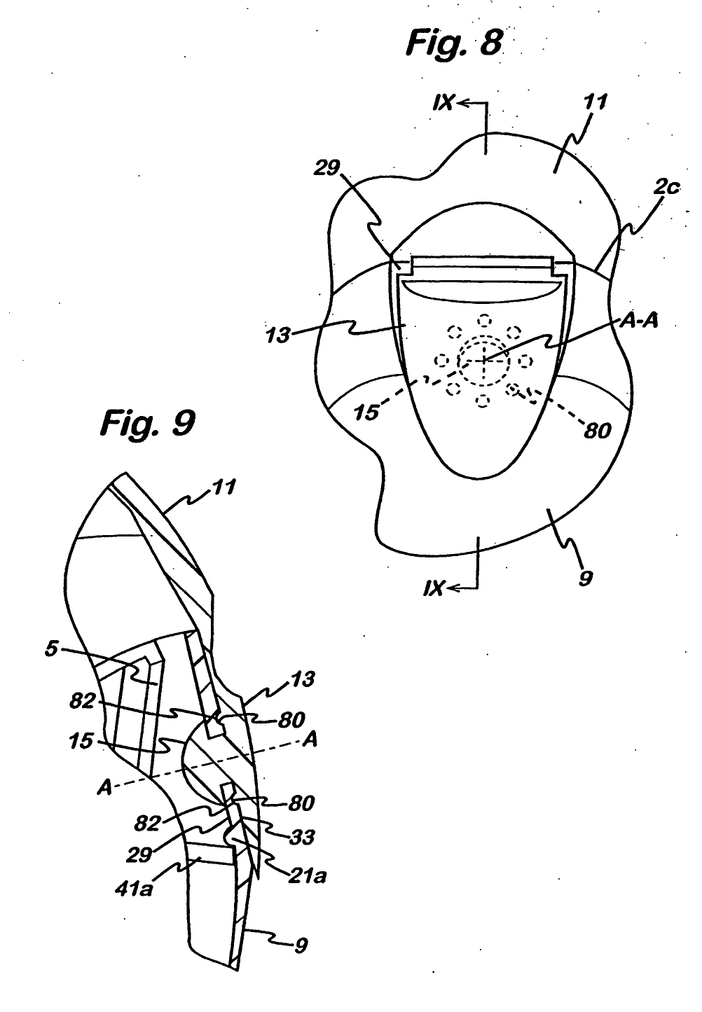

By way of example, a “ball-and-socket” indexing mechanism is shown in FIGS. 8 and 9 in which a circular array of protrusions 80 is formed on the inner surface of the hinge 13 co-axially with the knob 15, and a complementary circular array of sockets 82 is formed in the outer surface 33 of the bevel 29 on the container member 9 co-axially with the pivot axis A-A. When the protrusions 80 are located in the complementary sockets 82, the cover member 11 is indexed in one of a plurality of different predetermined angular positions it can adopt about the pivot axis A-A, for instance the first angular position of FIGS. 2 and 3 or the second angular position of FIGS. 4A and 4B. The cover member 11 is then able to be indexed in a new predetermined angular position by pivoting it about the pivot axis A-A to disconnect and re-engage the protrusions 80 and sockets 82, e.g. from the first angular position to the second angular position and vice-versa.

In an alternative embodiment, an indexing mechanism may be provided which indexes the cover member 11 in an end angular position between the first and second angular positions mentioned previously such that the cover member 11 does not cover the window 35 in the container member 9, thereby allowing the window 35 to be viewed by a user of the intranasal device 1.

Canister Unit

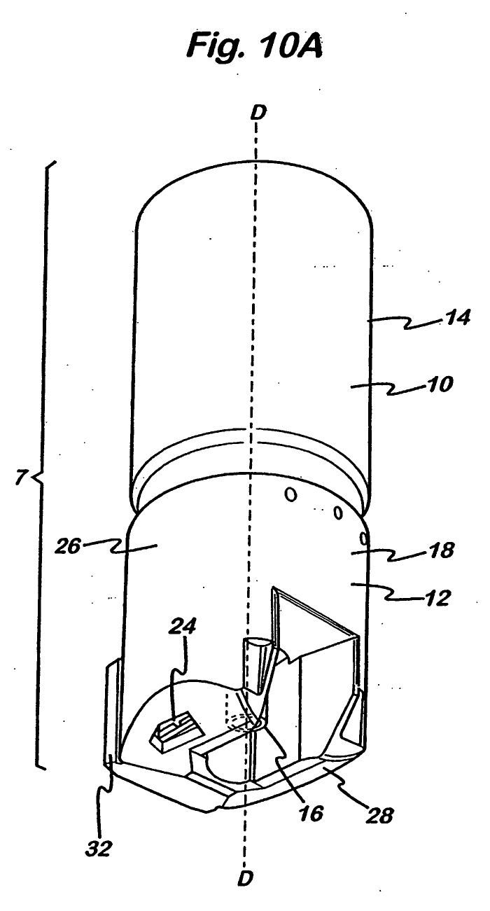

Turning attention now to FIGS. 10A to 10D, the canister unit 7 comprises an aerosol canister 10 of standard type which contains a drug formulated in a fluid propellant, e.g. a liquefied gas propellant such as a hydrofluoro alkane (HFA), for instance 1,1,1,2-tetrafluoroethane (CF₃CH₂F) (known as “HFA 134a”) or 1,1,1,2,3,3,3-heptafluoro-n-propane (CF₃CHFCF₃) (known as “HFA 227”) or a mixture thereof. The drug is typically for the treatment or prophylaxis of respiratory diseases or disorders, for example rhinitis. The drug may also be for the treatment or prophylaxis of other types of disease or disorder through systemic action of the drug. The canister unit 7 further comprises a dose counter head 12.

The canister 10 has a metal canister body 14 which, as known in the art, e.g. from metered dose inhalers (MDIs), has an open end which is capped by a valve assembly including a valve stem 16 which is mounted so it is movable relative to the canister body 14, between a retracted or open position relative to the canister body 14 in which the drug formulation is discharged from the canister 10 through the valve stem 16, and an extended or closed position relative to the canister body 14 in which the drug formulation is prevented from being discharged from the canister 10.

As known in the art, the valve assembly includes a biasing mechanism such as a spring (not shown) for biasing the valve stem 16 to the closed position. The valve assembly may be such as to further include a metering mechanism which operates so that a metered dose of the drug formulation is discharged when the valve stem 16 is in its open position. Typically, the valve assembly will have a metering chamber of fixed volume which in the closed position of the valve stem 16 is sealed from the external environment but in fluid communication with the canister body 14 whereby the metering chamber is filled with the drug formulation, and which in the open position of the valve stem is sealed from the canister body 14 and its contents, but placed in fluid communication with the external environment so that the metered dose of the drug formulation in the chamber is discharged to the external environment through the valve stem 16.

In this embodiment of the invention the valve assembly is a metering valve which dispenses a metered dose of the drug formulation per actuation thereof. A suitable metering valve is disclosed in WO98/29321, the content of which is hereby incorporated herein by reference.

The dose counter head 12 of the canister unit 7 has a hollow plastics body 18 of a plastics material which is fixedly secured to the canister 10 over the outlet end of the canister 10 having the valve assembly. The dose counter body 18 is fixed to the canister 10 to prevent it being taken off the canister 10, although it is free to rotate about a longitudinal axis D-D of the canister unit 7. The dose counter body 18 may be fixed to the canister 10 in the manner described and shown in WO01/28887 (Glaxo/Brand et al), the content of which is hereby incorporated herein by reference.

As best shown in FIG. 10D, the dose counter body 18 is formed with a display window 20. The dose counter head 12 further includes a dose counting mechanism (not shown) in the body 18 which, when actuated, advances a counter 22 thereof located in the window 20. When the counter 22 is advanced it results in the dose count shown thereby in the window 20 either being incremented to indicate the number of doses dispensed or, more preferably, decremented to show the number of doses left in the canister 10. The dose counter mechanism can take one of the forms described and shown in WO98156444 (Glaxo/Rand et al or Applicant's co-pending International patent application No. PCT/EP03/06466 (Applicant's Ref: PB60210), the contents of which are incorporated herein by reference.

An aperture 24 is provided in the outer surface of the dose counter body 18 to enable a driver to engage with the dose counting mechanism to advance the counter 22 when a dose of the drug formulation is dispensed from the canister 10 by the inner actuating part 5, as will be described in more detail hereinafter.

As shown in FIGS. 10A and 10B, the dose counter body 18 comprises a skirt-like lower section 26 and a U-shaped upper section 28. The dose counter body 18 also has a central opening 13 to the U-shaped upper section 28 through which the valve stem 16 protrudes. It will further be seen from FIGS. 10c and 10D that the dose counter window 20 is formed in a protrusion 32 in the outer peripheral surface of the U-shaped upper section 28.

Inner Actuating Part

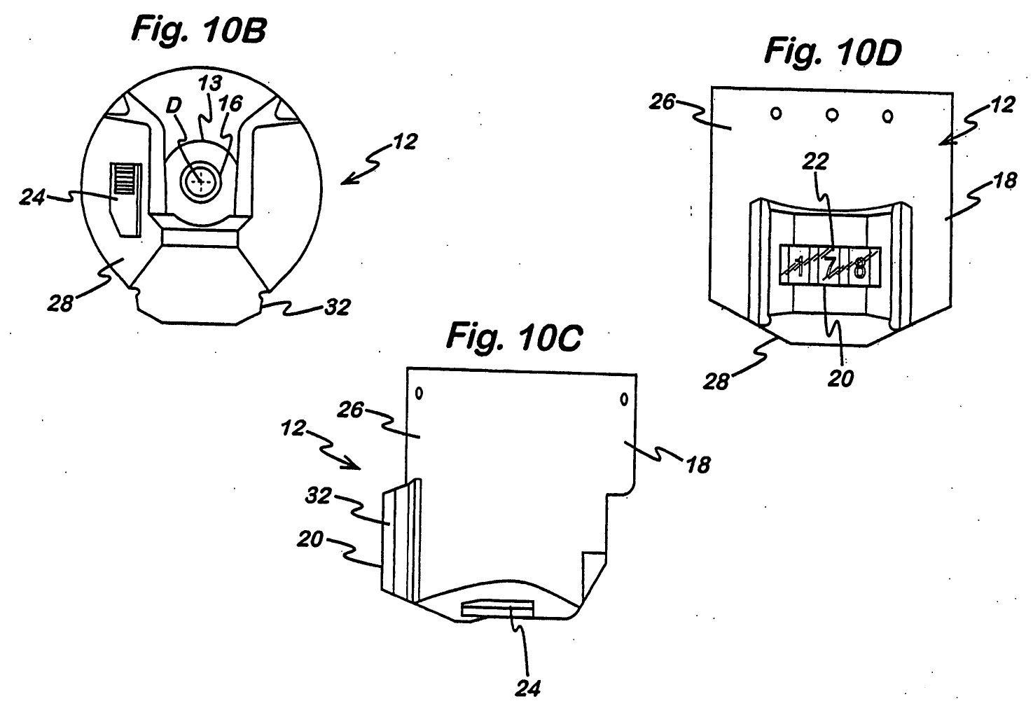

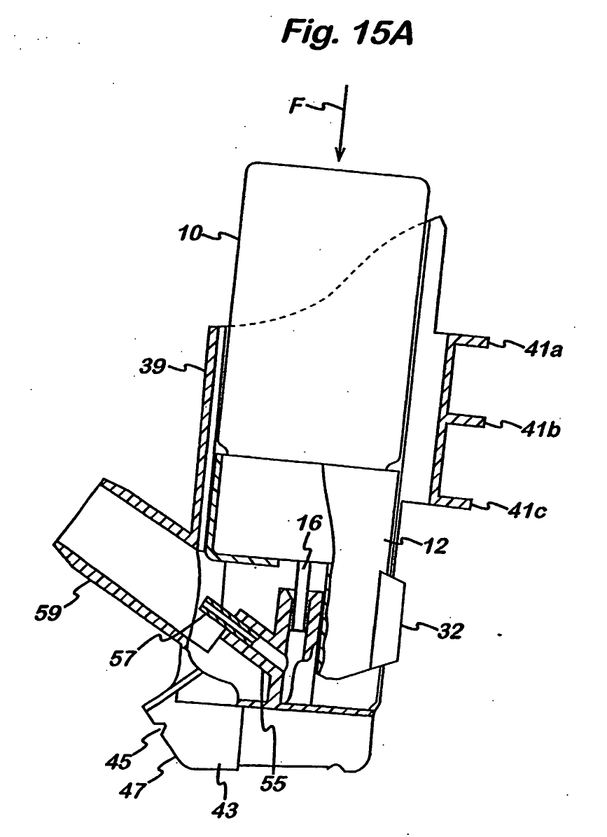

Attention is now turned to FIGS. 11 to 15B which show the inner actuating part 5. The inner actuating part 5 is of a plastics material, preferably polypropylene, made by a moulding process, preferably by injection moulding.

As will be seen, the inner actuating part 5 is of tubular construction having a main body 37 defining an axially-oriented cavity 38. The main body 37 has an outer surface 39 having a rear section 40 of shape and size which is complementary to the shape and size of the inner surface 19 of the container member cavity 17a, thereby enabling the inner actuating part 5 to fit snugly in the container member cavity 17a, as shown in FIGS. 3 and 7, for example. More particularly, the rear section 40 of the main body outer surface 39 is provided with a series of axially-spaced, circumferential ribs 41a-c which act as spacers to position the inner actuating part 5 along a vertical axis C-C in the container member 9, as shown in FIGS. 6 and 7. Moreover, as further shown in FIG. 7, the uppermost circumferential rib 41a is adapted to be snap fit underneath the locking rib 21a on the inner surface 19 of the container member 9.

FIGS. 11 to 13 show that the circumferential ribs 41a-c are intersected by longitudinal slots 42, 44. The longitudinal slots 42, 44 are positioned and sized so as to co-operate with the longitudinal ribs 27a, 27b on the inner surface 19 of the container member 9 of the outer casing part 3 to prevent rotation of the inner actuating part 5 in the container member 9 of the outer casing part 3.

It will further be seen that the inner actuating part 5 has a foot structure 43 which, as shown in FIG. 7, stands on a base surface 60 of the cavity 17a of the container member 9. The foot structure 43 includes a notch 45 on its forwardmost surface 47 which engages with the locking rib 21b at the lower end 25 of the cavity 17a of the container member 9. When the locking features 21a, 21b; 41a, 45 of the container member 9 and the inner actuating part 5 respectively engage with one another, the inner actuating part 5 is releasably fixed in place in the container member 9. Only when a sufficient separation force is applied is the inner-actuating part 5 released from the container member 9.

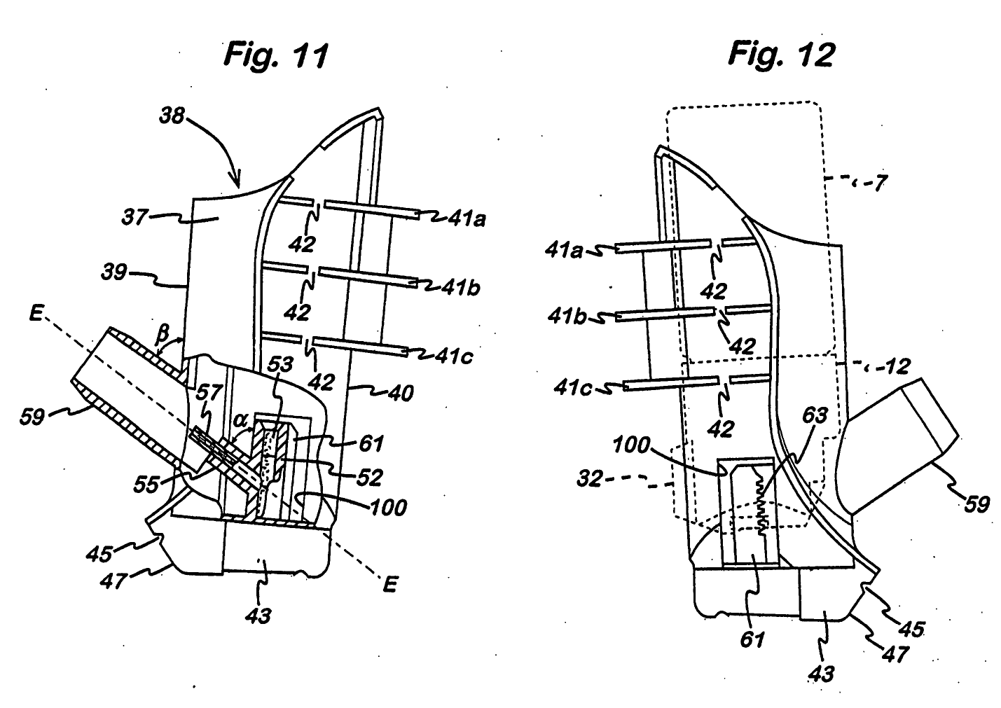

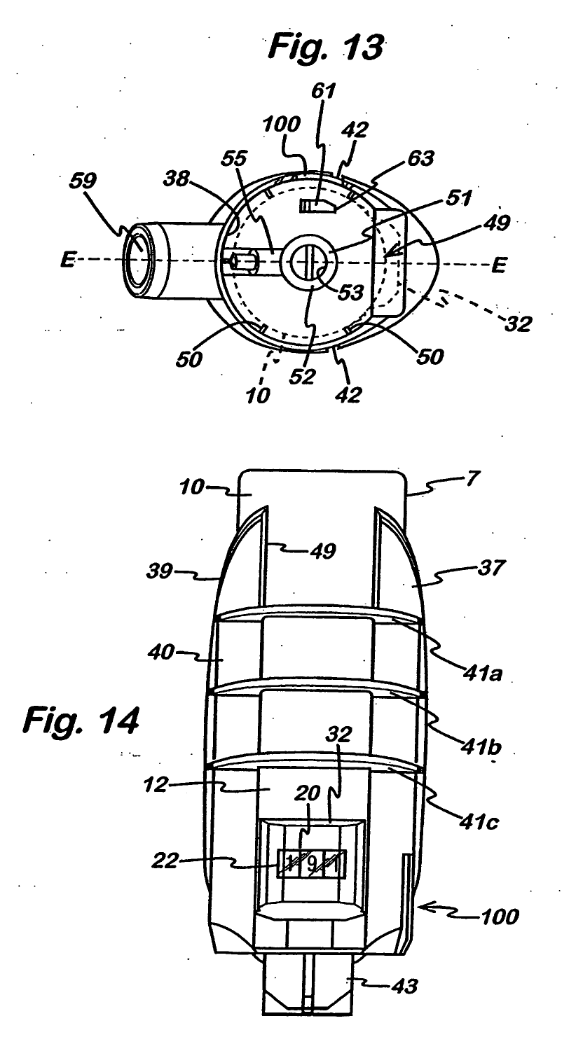

The rear section 40 of the main body 37 of the inner actuating part 5 has a longitudinally extending guide slot 49. The guide slot 49 is sized to slidingly receive the protrusion 32 on the dose counter body 18. The canister unit 7 can only be inserted into the inner actuating part 5 when the protrusion 32 is aligned with the guide slot 49 in the inner actuating part 5, as shown in FIG. 6. Thus, the guide slot 49 acts as a track along which the protrusion 32 slidingly moves to insert the canister unit 7 into the inner actuating part 5 and retract it therefrom. The guide slot 49 also co-operates with the protrusion 32 to act as an anti-rotation feature which prevents rotation of the dose counter head 12 in the inner actuating part 5, as will be understood by reference to FIGS. 12 and 13.

As shown in FIG. 13, the inner actuating part 5 also has longitudinal spacers 50 arranged circumferentially about the inner surface of the cavity 38 so that the canister unit 7 is generally co-axially mounted in the inner actuating part 5.

At the base of the cavity 38 of the inner actuating part 5 there is provided a hollow support 51 (so-called “stem block”) having a sleeve 52 with a bore 53 sized to receive the valve stem 16 of the canister 10. It will be appreciated that when the canister unit 7 is inserted axially into the cavity 38, the U-shaped upper section 28 of the dose counter head 12 encloses the support 51 oh three sides thereof. The hollow support 51 includes a hollow extension 55 which extends outwardly at an acute angle α to the outer periphery of the sleeve 52. The extension 55 has a bore 57 which opens into the bore 53 of the sleeve 52.

The bore 57 of the extension 55 is co-axial with a longitudinal axis E-E of a nozzle 59 which extends forwardly and upwardly at an acute angle β to the outer peripheral surface of the body 37 of the inner actuating part 5. The acute angles α, β may be the same, or substantially the same. As shown in FIG. 1, the nozzle 59 is shaped and sized to be received in a nostril 94 of a nose 96 of the patient 92.

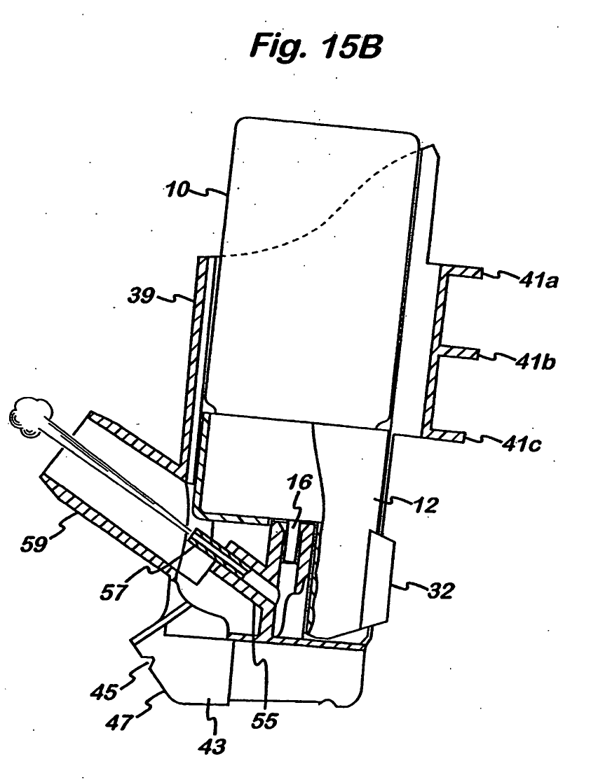

FIG. 15A shows the canister unit 7 mounted in the inner actuating part 5 with the valve stem 16 held in the bore 53 of the sleeve 52 of the hollow support 51. As will be understood by comparing FIGS. 15A and 15B, in operation the canister body 14 (and counter head 12) is depressed in the direction of arrow F relative to the inner actuating part 5 and the valve stem 16, the stem 16 being held stationary in the hollow support 51. This relative movement of the canister body 14 to the valve stem 16 causes a metered dose of the drug formulation to be discharged through the valve stem 16 into the bore 53 of the sleeve 52. The drug formulation is then channeled by the extension 55 into the nozzle 59 for delivery to the patient's nostril 94. The biasing mechanism in the valve assembly causes the canister body 14 to re-adopt the inoperative position shown in FIG. 15A on release of the depressing force F ready for the next actuation cycle.

It can therefore be seen from the above that the inner actuating part 5 contains all of the actuating elements for causing actuation of the canister 10 so that the drug formulation is discharged therefrom into the patient 92. In other words, the inner actuating part 5 is adapted to actuate the canister 10 when separate from the outer casing part 3.

Moreover, the base of the cavity 38 of the inner actuating part 5 is moulded with a rack-like post 61 having a serrated outer profile 63 (teeth) which co-operates with the dose counter mechanism such that on depression of the canister body 14 in the inner actuating part 5 to its operative position shown in FIG. 15B, the rack-like post 61 drives a cog system in the dose counter mechanism (see WO98/56444 and PCT/EP03/06466 supra) so that the counter 22 in the display 20 is advanced to reflect that the canister 10 has been actuated to dispense a dose of the drug-formulation.

It will be observed from FIGS. 11 to 13, for example, that the main body 37 of the inner actuating part 5 has an aperture 100 through a side thereof which is aligned with the rack-like post 61. The aperture 100 is left by that member of the inner actuating part mould assembly used to form the post 61. In this regard, it will be gathered from FIG. 13 that the post 61 does not register with the nozzle 59. That is to say, the post 61 is offset to the nozzle axis E-E such that it would not be possible to have the mould member for the post-61 extracted through the nozzle 59. This is because the nozzle diameter has to be small enough to be insertable into the patients nostril 94 and the post 61 is positioned to one side of the hollow support 51 which is in registration with the nozzle 59 for fluid communication therebetween.

As will be realised, the rack-like post 61 can be replaced by any other moulded dose counter mechanism driver structure as dictated by the form of the dose counter mechanism. Accordingly, the dose counter mechanism driver can take the form of another type of mechanical element, for example. Again, reference may be had to WO98/56444 supra.

It can be seen from FIG. 2 that once the canister unit 7 is assembled with the inner actuating part 5 and this assembly snap-fitted into the outer casing part 3, the outer casing part 3 acts as a protective casing for the canister unit 7 when in its closed state as it prevents actuation of the canister unit 7 and shields the nozzle 59 of the inner actuating part 5.

Operation

To use the intranasal device 1, the patient 92 moves the cover member 11 of the outer casing part 3 from the closed position shown in FIG. 2 to the open position shown in FIG. 3. The cover member 11 is then pivoted to the second angular position shown in FIG. 4A and then nested with the container member 9, as shown in FIG. 4B. Then, as shown in FIG. 1, the patient 92 grips the outer casing member 3 in one hand 90 and inserts the nozzle 59 into the nostril 94. The patient 92 then actuates the device 1 by depressing the canister body 14 into the inner actuating part 5 relative to the valve stem 16 with the index finger 98. This results in a metered dose of the drug formulation being delivered to the nostril.

Actuation of the device 1 can be confirmed by the patient 92 observing whether the dose counter 22 has been advanced (decremented/incremented). The patient 92 then closes the outer casing part 3 to protect the inner actuating part 5 and canister unit 7 until the next dose is required to be dispensed.

As will be seen from FIG. 1, when the outer casing part 3 is in its open state it acts as a holder/applicator for the patient 92 in the sense that the patient 92 is able to grip the outer casing part 3 in one hand 90 and to depress the canister body 14 (and counter head 12) into the inner actuating part 5 with the index finger 98 to cause a dose of the drug formulation to be dispensed through the nozzle 59 and the counter 20 to be incremented/decremented.

One of the numerous advantages of the intranasal device 1 is the use of separable outer casing and inner actuating parts 3, 5. Recalling that the inner actuating part 5 contains all of the functional features for actuating the canister unit 7, both from a drug delivery and dose counting point of view, this is the only part which needs to be tested and submitted for regulatory approval. The outer casing part 3 does not affect the performance of the inner actuating part 5 in any way. This would be in contrast to the case where the outer and inner parts 3, 5 are integrally formed. In this instance, any change to the external shape and configuration after approval would necessitate a new application for approval since the alteration may have an adverse effect on the internal functional features, especially if the component is moulded from a plastics material.

So, the inner actuating part 5 is able to be designed first and then the decorative outer protective casing part 3 designed afterwards. In this way, the inner actuating part 5 can be tested and submitted for regulatory approval before the outer casing part 3 is finalised. This shortens the lead time for developing an approved drug delivery device. Moreover, the outer casing part 3 can be re-designed to maintain a contemporary appearance etc. without requiring a new round of regulatory tests.

Appropriate drugs (or medicaments) for use in the present invention, for instance forming part of a pharmaceutical aerosol formulation having a fluid propellant, e.g. a HFA propellant, such as HFA 134a or HFA 227, may be selected from, for example, analgesics, e.g., codeine, dihydromorphine, ergotamine, fentanyl or morphine; anginal preparations, e.g., diltiazem; antiallergics, e.g., cromoglycate (e.g. as the sodium salt), ketotifen or nedocromil (e.g. as the sodium salt);—antiinfectives e.g., cephalosporins, penicillins, streptomycin, sulphonamides, tetracyclines and pentamidine; antihistamines, e.g., methapyrilene; anti-inflammatories, e.g., beclomethasone (e.g. as the dipropionate ester), fluticasone (e.g. as the propionate ester), flunisolide, budesonide, rofleponide, mometasone (e.g. as the furoate ester), ciclesonide, triamcinolone (e.g. as the acetonide), 6α,9α-difluoro-11β-hydroxy-16α-methyl-3-oxo-17α-propionyloxy-androsta-1,4-diene-17β-carbothioic acid S-(2-oxo-tetrahydro-furan-3-yl) ester or 6α,9α-Difluoro-17α-[(2-furanylcarbonyl)oxy]-11β-hydroxy-16α-methyl-3-oxo-androsta-1,4-diene-17β-carbothioic acid S-fluoromethyl ester; antitussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl]sulfonyl]ethyl]amino]ethyl-2(3H)-benzothiazolone; PDE4 inhibitors e.g. cilomilast or roflumilast; leukotriene antagonists e.g. montelukast, pranlukast and zafirlukast; [adenosine 2a agonists, e.g. 2R,3R,4S,5R)-2-[6. Amino-2-(1S-hydroxymethyl-2-phenyl-ethylamino)-purin-9-yl]-5-(2-ethyl-2H-tetrazol-5-yl)-tetrahydro-furan-3,4-diol (e.g. as maleate)]; [α4 integrin inhibitors e.g. (2S)-3-[4-({[4-(aminocarbonyl)-1-piperidinyl]carbonyl}oxy)phenyl]-2-[((2S)-4-methyl-2-{[2-(2-methylphenoxy) acetyl]amino}pentanoyl)amino] propanoic acid (e.g. as free acid or potassium salt)], diuretics, e.g., amiloride; anticholinergics, e.g., ipratropium (e.g. as bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagons. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament and/or to minimise the solubility of the medicament in the propellant.

Preferably, the medicament is for the treatment of inflammatory and/or allergic conditions of the nasal passages such as rhinitis, e.g. seasonal and perennial rhinitis as well as other local inflammatory conditions such as asthma, COPD and dermatitis. Preferably, the medicament is an anti-inflammatory compound for the treatment of asthma or rhinitis.

It is to be understood that the exemplary embodiment of the present invention outlined above are for the purposes of illustration only, and that the invention can be modified, varied and take on other guises within the scope of the appended claims. Mindful of this, the use of reference numerals in the claims is not to be taken as having a limiting effect on the scope of the claims.

For the avoidance of doubt, the use herein of terms such as “generally”, “substantially”, “about” and the like when referring to a parameter of the invention is meant to include the absolute parameter.

1. A holder for holding a dispensing container system which is adapted to dispense a quantity of a fluid contained therein on movement thereof relative to the holder and further includes a dispensing counter means for counting the number of quantities of the fluid dispensed, the holder having a moulded plastics body with inner and outer surfaces, the inner surface bounding a cavity adapted to receive the dispensing container system in movable relation thereto, the cavity having moulded counter advance means adapted in use to co-operate with the dispensing counter means on relative movement between the dispensing container system and the body to advance the dispensing counter means to indicate the dispensing of a quantity of the fluid, wherein the body is formed with an outlet port in communication with the cavity such that the fluid dispensed from the dispensing container system is dischargeable therethrough, and wherein an aperture extends through the body from the outer surface to the inner surface in alignment with the counter advance means.

2. The holder of claim 1 in which the counter advance means is a rack-like means.

3. The holder of claim 2 in which the rack-like means is a rack-like member or post.

4. The holder of claim 1 in which the cavity extends along an axis of the holder and the dispensing container system is axially, slidably movable in the cavity to dispense therefrom.

5. The holder of claim 1 in which the body is adapted to receive the dispensing container system in the cavity only when it is arranged in a predetermined orientation about an axis thereof.

6. The holder of claim 5 in which the axis of the dispensing container system extends between a trailing end and a leading end of the dispensing container system.

7. The holder of claim 6 wherein the counter means is located at the leading end and the body is adapted to prevent rotation of the counter means in the cavity from the predetermined orientation.

8. The holder of claim 5 in which the body is provided with an alignment feature for aligning the system in the predetermined orientation.

9. The holder of claim 8 in which the alignment feature is a slot in the body along which the system is slidable.

10. The holder of claim 9 in which the slot is adapted to engage the counter means to prevent rotation thereof in the cavity.

11. The holder of claim 1 any one of the preceding claims in which the counter means has a display part for displaying indicia to indicate the count and the body has a window for registering with the display part.

12. The holder of claim 11 in which the body is adapted to receive the dispensing container system in the cavity only when it is arranged in a predetermined orientation about an axis thereof, in which the body is provided with an alignment feature for aligning the system in the predetermined orientation and wherein the window forms part of the alignment feature.

13. The holder of claim 1 in which the outlet port is in the form of a nozzle.

14. The holder of claim 13 in which the nozzle is sized to be received in a nostril of a nose of a human patient.

15. The holder of claim 1 wherein the counter advance means is non-aligned with the outlet port.

16. The holder of claim 1 further having a hollow stand structure with a passageway in fluid communication with the outlet port, an outlet member of the container system through which fluid is dispensed being receivable in the passageway so that the passageway is able to channel fluid dispensed from the outlet member to the outlet port.

17. The holder of claim 16 wherein the passageway has an entrance opening into which the outlet member is insertable, and an exit opening aligned with the outlet port.

18. The holder of claim 17 wherein the exit opening is spaced from the outlet port by a void area.

19. The holder of claim 1 wherein the counter advance means is located on a base surface of the cavity.

20. The holder of claim 19 further having a hollow stand structure with a passageway in fluid communication with the outlet port, an outlet member of the container system through which fluid is dispensed being receivable in the passageway so that the passageway is able to channel fluid dispensed from the outlet member to the outlet port, wherein the stand structure is on the base surface of the cavity.

21. The holder of claim 19 wherein the counter advance means is positioned to a side of the stand structure.

22. The holder of claim 1 wherein the body is formed by injection moulding.

23. The holder of claim 1 including the dispensing container system.

24. The holder of claim 23 in which the system comprises a container to which the dispensing counter means is secured.

25. The holder of claim 23 wherein the container has an outlet member and a container member which contains the fluid and is movable relative to the outlet member to dispense the fluid from the outlet member.

26. The holder of claim 25 further having a hollow stand structure with a passageway in fluid communication with the outlet port, an outlet member of the container system through which fluid is dispensed being receivable in the passageway so that the passageway is able to channel fluid dispensed from the outlet member to the outlet port, in which the container system dispenses when the container member is moved relative to the outlet member and the stand structure is adapted to hold the outlet member stationary relative to the body so that the container member is movable in the body relative to the outlet member.

27. The holder of claim 25 in which the body is adapted to receive the dispensing container system in the cavity only when it is arranged in a predetermined orientation about an axis thereof, in which the axis of the dispensing container system extends between a trailing end and a leading end of the dispensing container system, and in which the outlet member is at the leading end of the container system.

28. The holder of claim 23 wherein the container is an aerosol container.

29. The holder of claim 28 including the dispensing container system, wherein the container has an outlet member and a container member which contains the fluid and is movable relative to the outlet member to dispense the fluid from the outlet member, and wherein the outlet member forms a part of a valve mechanism secured to the container member which is selectively openable by movement of the container member in the body relative to the outlet member.

30. The holder of claim 23 wherein the container system has a metering mechanism for dispensing metered doses of the fluid and the dispensing counter means is adapted to count the number of doses dispensed.

31. The holder of claim 29 wherein the valve mechanism is a metering valve.

32. The holder of claim 23 wherein the fluid is a drug composition.

33. The holder of claim 1 in which the body is moulded in a mould having a mould part which moulds the counter advance means, the mould part being arranged in the mould so that it leaves the aperture in the body to enable its extraction from the body after the body has been moulded.

34. A holder assembly for a dispensing container system having an outer part and an inner part in the form of the holder of any one of the preceding claims, the inner part being receivable in the outer part in an operational position in which the aperture is covered by the outer part.

35. The holder assembly of claim 34 wherein the outer part is movable between a closed state, in which the outer part is able to encapsulate the inner part when in the operational position and a dispensing container system held by the inner part, and an open state, in which the dispensing container system is uncovered thereby enabling it to be moved relative to the inner part for dispensing a quantity of fluid therefrom and advancement of the dispensing counter means.

36. (canceled)
